(19) United States
(12) Patent Application Publication
(10) Pub. No.: US 2008/0233630 A1
(43) Pub. Date: Sep. 25, 2008

(54) Apparatus and Method for Magnetically Separating Cells From Mixture

(76) Inventors: Young-Ho Kim, Seoul (KR); Il-Joo Shim, Gwacheon-si (KR); Myeong-Se Jung, Seoul (KR); Byeong-Gil Lee, Gyeonggi-do (KR)

Correspondence Address: LOWE HAUPTMAN HAM & BERNER, LLP, 1700 DIAGONAL ROAD, SUITE 300, ALEXANDRIA, VA, 22314, US

(21) Appl. No.: 10/585,860

(22) PCT Filed: Mar. 23, 2006
(86) PCT No.: PCT/KR06/01077

§ 371 Date: Jul. 11, 2006

(30) Foreign Application Priority Data
Jun. 20, 2005 (KR) 10-2005-0052880
Sep. 27, 2005 (KR) 10-2005-0089607
Nov. 14, 2005 (KR) 10-2005-0108353
Nov. 14, 2005 (KR) 10-2005-0108355
Nov. 14, 2005 (KR) 10-2005-0108357

(51) Int. Cl. C12N 1/00; C12M 1/42

(52) U.S. Cl. 435/243; 435/283.1

(57) ABSTRACT

According to the conventional process of separating necessary cells from a cell mixture containing specific cells tagged with magnetic beads, there is a problem in that separation process is complicated when the separation efficiency is high whereas the separation efficiency is relatively low when the separation process is simplified. The present invention provides an apparatus and method for separating cells from a cell mixture simply and efficiently through the processes of creating a cell mixture layer between upper and lower plates by adjusting a gap between the upper and lower plates and of adjusting a thickness of the created layer and separating the layer while applying a magnetic field to the layer from the upper plate, wherein the lower plate is provided with a cell mixture holding portion, in which the cell mixture containing specific cells tagged with magnetic beads is accommodated in an upwardly convex shape, at a top surface thereof and the upper plate is positioned above the lower plate to face each other and to adsorb the cell mixture accommodated in the cell mixture holding portion of the lower plate into a bottom surface thereof.

### TECHNICAL FIELD

The present invention relates to an apparatus and method for separating cells from a cell mixture, and more particularly, to an apparatus and method for separating necessary cells from a cell mixture by applying a magnetic field to the cell mixture in which specific cells tagged with magnetic carriers are mixed.

### BACKGROUND ART

In U.S. Pat. No. 6,602,422 (entitled “micro column system”), if a magnetic field is applied to a column from the outside while a cell mixture in which specific cells tagged with magnetic beads (serving as a magnetic carrier) are mixed is poured into the column with small steel balls filled therein in a gravitational direction, the specific cells are adsorbed while flowing through gaps between the magnetized steel balls but the other cells flow downward. At this time, the specific cells which have been bound to the steel balls and thus separated from the mixture are recovered by eliminating the applied magnetic field and then causing a buffer solution to flow.

However, when a user intends to separate the cells with a variety of sizes using the column type cell separation apparatus, the used steel balls should be changed in accordance with the sizes of magnetic beads or specific cells and the adsorption intensity for the specific cells to be separated since the movement of cells may be hindered by the size of gaps between the steel balls.

Further, since the cells mixed in the cell mixture gather around the gaps caused by the accumulated steel balls, it may be difficult to recover the specific cells separated from the mixture.

Furthermore, in order to prevent the gaps between the steel balls from being choked as the cells coagulate, the steel balls in the cell separation apparatus should be periodically shaken to be homogeneous using a pipette during the separation process.

In U.S. Pat. No. 5,602,042 (entitled “method and apparatus for magnetically separating biological particles from a mixture”), biological particles tagged with beads are separated from a mixture by adhering to a plate by means of an applied magnetic field while a separation means including a magnet and a plate is vertically moved and rotated in a state where the separation means is immersed in the mixture with which a closed container.

However, in a case where biological particles are separated using the container type cell separation apparatus, other biological particles adhering to an outer side of a housing of the separation means are collected onto the plate while taking the separation means out of the mixture. Thus, its separation efficiency will be greatly lowered.

In addition, since the biological particles are separated in a state where the mixture is filled in the closed container, a large amount of samples are needed to thereby incur high costs and the separation means for implementing a vertical and rotational moving mechanism is complicated. Further, since an exposed portion such as a housing of the separation means is brought into direct contact with the mixture, the possibility of contamination of the mixture will be increased.

In Korean Patent Application No. 2004-25421 (entitled “apparatus and method for isolating cells using droplet type cell suspension”), therefore, a cell mixture containing specific cells tagged with magnetic beads is formed into a droplet type cell mixture and a magnetic field is then applied to the droplet type cell mixture such that the cell mixture is divided into the specific cells positioned at an upper portion thereof and the other cells positioned at a lower portion thereof. Then, only a buffer solution is additionally supplied to the droplet type cell mixture to completely isolate the lower other cells by means of gravity and then to recover the isolated specific cells. Accordingly, the configuration and process of the cell isolation apparatus can be simplified.

However, in a case where the specific cells are isolated using the droplet type cell mixture, the configuration and process of an apparatus for forming the cell mixture into a droplet type cell mixture are complicated.

Further, since the cell mixture is suspended and thus exposed to the air, the lower cells other than the specific cells may fluctuate. Furthermore, when the buffer solution is additionally added to remove the lower cells other than the specific cells to be isolated, a portion of the lower other cells may be again mixed with the upper specific cells. Thus, there is a problem in that isolation efficiency is lowered.

On the other hand, in a case where the other cells are bound to a surface where the specific cells are recovered, it may have a bad influence on the subsequent test in which the recovered specific cells will be used. Therefore, the purity of the obtained specific cells needs to be enhanced to the utmost.

### DISCLOSURE OF INVENTION

### Technical Problem

An object of the present invention is to provide an apparatus and method for separating cells in a simple and highly efficient manner through the process of creating a cell mixture layer by adjusting a gap between an upper plate and a lower plate oppositely positioned below the upper plate to contain a cell mixture between the upper and lower plates and adjusting a width of the formed cell mixture layer or separating the cell mixture layer while applying a magnetic field to the upper plate over the cell mixture layer.

### Technical Solution

According to an aspect of the present invention for achieving the object, there is provided a cell separation apparatus, comprising a lower plate provided with a cell mixture holding portion, in which a cell mixture containing specific cells tagged with magnetic carriers is accommodated in an upwardly convex shape, at a top surface thereof; an upper plate positioned above the lower plate to face each other and to adsorb the cell mixture accommodated in the cell mixture holding portion of the lower plate into a bottom surface thereof; a magnetic field applying means positioned on a top surface of the upper plate; and a gap adjusting means coupled to the upper or lower plate to adjust a gap between the upper and lower plates to be increased or decreased, wherein the gap between the upper and lower plates is decreased by the gap adjusting means such that the cell mixture accommodated in the cell mixture holding portion is adsorbed in the bottom surface of the upper plate and then formed into a cell mixture layer, and the gap between the upper and lower plates is increased by the gap adjusting means such that the specific cells moved toward the upper plate by means of a magnetic field applied to the created cell mixture layer through the magnetic field applying means and cells other than the specific cells moved toward the lower plate by means of gravity are divided and then positioned in the bottom surface of the upper plate and the cell mixture holding portion of the lower plate, respectively.

Preferably, a cell mixture adsorbing portion is formed at the bottom surface of the upper plate such that the cell mixture holding portion of the lower plate is positioned to correspond to the cell mixture adsorbing portion of the upper plate.

The cell separation apparatus of the present invention may further comprise an upper housing with an open bottom and a lower housing with an open top, wherein the magnetic field applying means and the upper plate are installed within the upper housing such that the magnetic field applying means is positioned on the top surface of the upper plate, the lower plate and the gap adjusting means are installed within the lower housing such that the gap adjusting means is coupled to the lower plate, and the lower plate is vertically moved by the gap adjusting means to adjust the gap between the upper and lower plates in a state where the upper housing is coupled with the lower housing such that the bottom surface of the upper plate and the cell mixture holding portion of the lower plate are positioned to face each other.

Preferably, the gap adjusting means comprises a lower plate support formed with a recess for accommodating the lower plate therein at a top side thereof and a bolt-sh aped connection at a bottom side thereof, and a lower plate support moving dial having a nut-shaped connection threadedly engaged with the bolt-shaped connection of the lower plate support; and the lower plate support is vertically moved by turning or rotating the lower plate support moving dial.

Preferably, the gap adjusting means further comprises a dial stopper for restricting the rotation of the lower plate support moving means such that the cell mixture layer can be maintained.

Preferably, the dial stoppers are installed on a bottom surface of the lower plate support moving dial and a predetermined portion of the lower housing such that the dial stopper is brought into contact with the bottom surface of the lower plate support moving dial at a position where the rotation of the lower plate support moving dial should be prevented.

Preferably, the gap adjusting means comprises a lower plate support formed with a recess for accommodating the lower plate therein at a top side thereof and a roller at a bottom side thereof, a lower plate support moving bar for vertically moving the lower plate support in such a manner that the roller of the lower plate support is brought into contact with a plurality of steps with different levels decreasing from one side to another side, and bar moving dial having a pinion portion meshed with a rack portion formed on a side surface of the lower plate support moving bar; and the lower plate support is vertically moved as the level of the steps of the lower plate support moving bar brought into contact with the roller of the lower plate support is changed by turning or rotating the bar moving dial.

Further, a groove for temporarily restricting a motion of the lower plate support may be formed on each of the steps.

Preferably, the lower plate support moving bar includes a lower plate support shaking portion further extending from the step with the highest level and formed with a plurality of grooves.

The cell separation apparatus of the present invention may further comprise a housing, wherein the upper and lower plates are installed within the housing such that the bottom surface of the upper plate and the cell mixture holding portion of the lower plate are positioned to face each other; the magnetic field applying means is positioned on the top surface of the upper plate; the gap adjusting means is coupled with the upper plate; and the upper plate is vertically moved by the gap adjusting means to adjust the gap between the upper and lower plates.

Preferably, the gap adjusting means further comprises a stopper for restricting an upward motion of the upper plate support to allow the cell mixture layer to be maintained.

According to another aspect of the present invention for achieving the object, there is provided a cell separation method comprising the steps of (a) creating a cell mixture containing specific cells tagged with magnetic beads into a cell mixture layer by adjusting a gap between upper and lower plates to be decreased such that the cell mixture which is accommodated in a cell mixture holding portion of the lower plate in an upwardly convex shape can be adsorbed in a bottom surface of the upper plate positioned opposite to the cell mixture holding portion of the lower plate; (b) moving the specific cells toward the upper plate by applying a magnetic field to the cell mixture layer created in step (a) from the upper plate and simultaneously moving cells other than the specific cells toward the lower plate by means of gravity; and (c) allowing the specific cells moved toward the upper plate and the other cells moved toward the lower plate in step (b) to be divided and then positioned in the bottom surface of the upper plate and the cell mixture holding portion of the lower plate, respectively, when the cell mixture layer is separated by increasing the gap between the upper and lower plates.

The cell separation method of the present invention may further comprise the step of, after step (a), adjusting the gap between the upper and lower plates to maintain a thickness of the cell mixture layer at an optimal cell separation state.

Further, the cell separation method of the present invention may further comprise the steps of (d1) creating a specific cell mixture layer by decreasing the gap between the upper and lower plates after removing the other cells divided and positioned in the lower plate in step (c) or replacing the lower plate with a new one and then injecting a buffer solution containing no cells in the lower plate; (e1) homogenizing the specific cell mixture layer by changing the gap between the upper and lower plates repeatedly several time while maintaining the specific cell mixture layer created in step (d1) (f1) moving the specific cells toward the upper plate by the magnetic field applied to the specific cell mixture layer homogenized in step (e1) from the upper plate and simultaneously moving the other cells in the specific cell mixture layer toward the lower plate by means of gravity; and (g1) allowing the specific cells moved toward the upper plate and the other cells moved toward the lower plate in step (f1) to be divided and then positioned in the bottom surface of the upper plate and the cell mixture holding portion of the lower plate, respectively, when the specific cell mixture layer is separated by increasing the gap between the upper and lower plates.

Furthermore, the cell separation method of the present invention may further comprise the steps of (d2) creating an other cell mixture layer by decreasing the gap between the upper and lower plates after removing the specific cells divided and positioned in the upper plate in step (c) or replacing the upper plate with a new one and then additionally injecting a buffer solution containing no cells in the lower plate; (e2) homogenizing the other cell mixture layer by changing the gap between the upper and lower plates repeatedly several times while maintaining the other cell mixture layer created in step (d2) (f2) moving the specific cells toward the upper plate by the magnetic field applied to the other cell mixture layer homogenized in step (e2) from the upper plate and simultaneously moving the other cells in the other cell mixture layer toward the lower plate by means of gravity; and (g2) allowing the specific cells moved toward the upper plate and the other cells moved toward the lower plate in step (f2) to be divided and then positioned in the bottom surface of the upper plate and the cell mixture holding portion of the lower plate, respectively, when the other cell mixture layer is separated by increasing the gap between the upper and lower plates.

### ADVANTAGEOUS EFFECTS

According to the cell separation apparatus and method of the present invention, necessary cells can be separated through a process of creating a cell mixture layer by adjusting a vertical gap of the cell separation chip composed of an upper plate and a lower plate for accommodating a cell mixture and of adjusting a thickness of the created cell mixture layer and separating the layer. Therefore, all the cells can be separated using the same cell separation chip regardless of the size of cells. Further, the separation process can be performed without any additional processes such as a pipetting process, a rotating process and a buffer solution injection process. Furthermore, separation efficiency can be enhanced by adjusting the thickness of the cell mixture layer in accordance with the separating conditions while applying a relatively strong magnetic field to the cells. Moreover, the separation efficiency can be further enhanced by additionally removing unnecessary cells through the homogenization process performed after the separation of the necessary cells.

### BRIEF DESCRIPTION OF THE DRAWINGS

FIG. 1 is an exploded perspective view of an apparatus for separating cells by applying a magnetic field to a cell mixture layer in which specific cells tagged with magnetic beads are mixed according to an embodiment of the present invention.

FIG. 2 is a view showing a state where upper and lower plates are not mounted to the cell separation apparatus according to the embodiment of the present invention.

FIG. 3 is a view showing a state where a cell mixture is contained in the lower plate of the cell separation apparatus according to the embodiment of the present invention.

FIG. 4 is a view showing a state where the cell mixture contained in the lower plate shown in FIG. 3 is formed into the cell mixture layer and an upper body is then covered onto a lower body in order to start the separation of cells.

FIGS. 5 to 8 are views illustrating the operation of the cell separation apparatus and the state of the cell mixture when the cells are separated in the cell separation apparatus according to the embodiment of the present invention.

FIG. 9 is a view showing the state of the upper and lower plates when the upper body has been separated from the lower body after the process of FIG. 8.

FIG. 10 is a view showing a state where cells other than the specific cells in the lower plate are removed and a new solution is then added into the lower plate to enhance the purity of the specific cells remaining in the upper plate.

FIGS. 11 to 18 are views each illustrating a process of causing a solution of the specific cells to be in a homogeneous state and then separating the specific cells after the upper and lower bodies are coupled with each other in a state of FIG. 10.

FIG. 19 is an exploded perspective view of an apparatus for separating cells by applying a magnetic field to a cell mixture layer in which specific cells tagged with magnetic beads are mixed according to another embodiment of the present invention.

FIGS. 20 to 22 are views each illustrating a process of separating the cells using the cell separation apparatus according to another embodiment of the present invention.

FIG. 23 is a view showing a state where the specific cells remaining in the upper plate are removed and a proper amount of a new solution is added into the lower plate to enhance the purity of the lower other cells remaining in the lower the plate after the process of FIG. 22.

FIGS. 24 to 28 are views each illustrating a process of causing a solution of the other cells to be in a homogeneous state and then separating the other cells after the upper and lower bodies are coupled with each other in a state of FIG. 23.

FIGS. 29 and 30 are perspective views showing an apparatus for separating cells by applying a magnetic field to a cell mixture layer in which specific cells tagged with magnetic beads are mixed according to a further embodiment of the present invention.

### BEST MODE FOR CARRYING OUT THE INVENTION

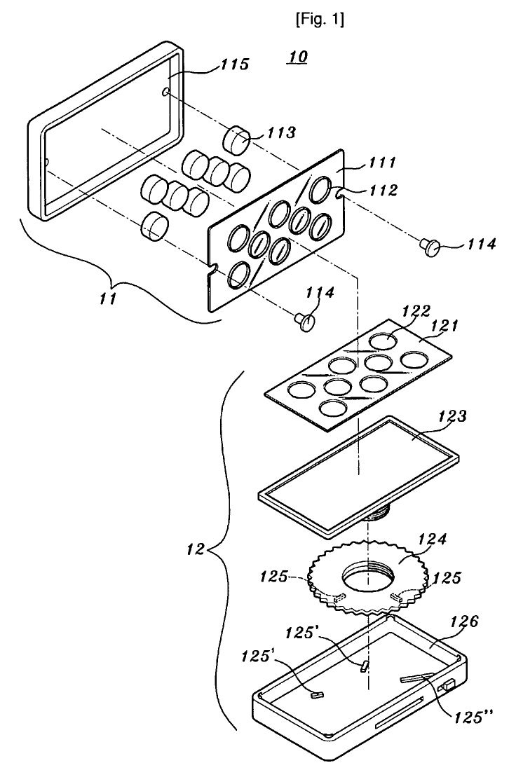

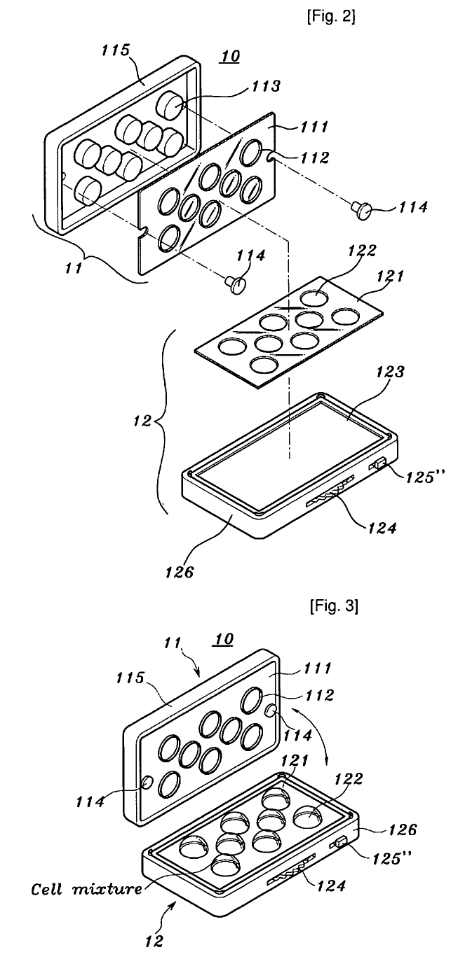

As shown in FIGS. 1 to 4, a cell separation apparatus 10 according to an embodiment of the present invention comprises an upper body 11 and a lower body 12. Here, a process of separating cells from a cell mixture is executed in a state where the upper body 11 for causing a magnetic field to be applied to the cell mixture is covered onto and then integrally coupled with the lower body 12 containing the cell mixture.

The upper body 11 includes an upper plate 111, magnets 113 positioned at an upper surface of the upper plate 111 to apply a magnetic field, an upper housing 115 with an open bottom for accommodating the upper plate 111 and the magnets 113 therein, and upper plate fixing means 114 for fixing the upper plate 111 to the upper housing 115.

The upper plate 111 includes a plurality of cell mixture adsorbing portions 112 formed on a lower surface of the upper plate to adsorb a cell mixture from the lower body 12. The lower plate 111 may be formed into a flat and transparent chip with a thickness of 3 mm or less.

Each of the cell mixture adsorbing portions 112 may be formed either into a recess when the upper plate 111 is made of a hydrophilic biocompatible material such as polymethylmethacrylate (PMMA), polypropylene or polyimide or into a ring when the upper plate is made of a hydrophobic biocompatible material such as polydimethylsiloxane (PDMS). In such a case, the cell mixture is adsorbed into the recess or the circumference of the adsorbed cell mixture is enclosed by the ring to define the boundary of the cell mixture.

Each of the magnets 113 is installed in a state where they are accommodated in the upper housing 115. Each of the magnets 113 is preferably positioned to correspond to the cell mixture adsorbing portion 112 such that the magnetic field generated from the relevant magnet can be concentrated on the relevant cell mixture adsorbing portion 112. In such a case, the size of the magnet 113 is equal to or slightly smaller than that of the cell mixture adsorbing portion and the thickness of the cell mixture adsorbing portion 112 is formed smaller than that of the upper plate 111 with a thickness of 3 mm or less such that the magnetic field can be sufficiently exerted thereon.

In order to move specific cells tagged with magnetic beads in the cell mixture contained in the lower body toward the upper plate 111, the magnetic field generated in the magnet 113 should be maintained at a certain level enough to overcome the gravity exerted on the specific cells.

In the embodiments of the present invention, each of the magnets is made of a neodymium permanent magnet such that it can generate a relatively strong magnetic field with a strength of approximately 0.5 T while occupying a minimum space. Accordingly, the compact cell separation apparatus with high separation efficiency can be obtained.

The upper plate fixing means 114 allows the upper plate 111 to be fixed to the upper housing 115 in a state where an upper surface of the fixing means faces the magnet 113.

The lower body 12 includes a lower plate 121, a lower plate support 123 for holding the lower plate 121 to perform the separation process in such a state where the lower plate 121 is securely seated in the lower plate support, a lower plate support moving dial 124 which rotates to cause the lower plate support 123 to move in a vertical direction, and a lower housing 126 with an open top for accommodating the lower plate 121, the lower plate support 123 and the lower plate moving dial 124 therein.

Dial stopper 125, 125′ and 125″ for restricting the lower plate support moving dial from being rotated beyond a certain limit are installed at predetermined positions on a bottom surface of the lower plate support moving dial 124 and corresponding positions on a floor surface of the lower housing 126.

The lower plate 121 is configured such that a cell mixture to be separated is received in a cell mixture holding portion 122 formed in a top surface thereof in a convex shape. The lower plate 121 may be formed into a flat and transparent chip with a thickness of 3 mm or less. In such a case, it is preferred that the lower plate be used together with the upper plate 111 as a set of a cell separation chip and also be discarded after one time use.

Each of the cell mixture holding portions 122 is shaped in a circular or similar form. A surface of the lower plate 121 is coated or recessed such that an angle of contact between a liquid surface and a solid surface may be maintained at about 90 degrees or more when a liquid is placed onto the level solid surface. Then, a certain amount of cell mixture is injected and received in the cell mixture holding portion 122 such that the received cell mixture takes the shape of an upward hemisphere.

Here, a case where the cell mixture holding portion 122 is formed into a recess will be discussed. If the lower plate 121 is made of a hydrophilic material, no additional treatment is required since the cell mixture does not flow out of the recess. However, if the lower plate 122 is made of a hydrophobic material, an inner surface of the recess should be coated with a hydrophilic material to allow the cell mixture to be easily adsorbed.

The cell mixture holding portion 122 is formed at a position corresponding to the cell mixture adsorbing portion 112 of the upper plate 111 to create a layer while the cell mixture received in the cell mixture holding portion is adsorbed to the cell mixture adsorbing portion 112.

The layer of the adsorbed cell mixture is created into a space composed of a liquid to which magnetic field and gravity are applied upward and downward, respectively. That is, the layer provides an environment in which the specific cells tagged with magnetic beads and the other cells are clearly separated from each other by the opposite forces.

In a case where the cell mixture layer is formed between the cell mixture adsorbing portion 112 and the cell mixture holding portion 122 to separate the cells, the cell separation process is performed while a maximum magnetic field is applied to the cell mixture adsorbing portion 112 in a state where the cell mixture is not in a dynamic condition but in a static condition. Therefore, the cell separation process is simplified and its separation efficiency is increased. Further, since the cell mixture is consumed as much as required in the cell separation, the separation costs can also be reduced.

The amount of cells to be separated can be determined by adjusting the number or size of the cell mixture adsorbing portions 112 of the upper plate 111 and the cell mixture holding portions 122 of the lower plate 121 which create the cell mixture layer between the portions. In addition, since several kinds of the cells can be separated simultaneously, the cell separation suitable to the various circumstances can be made.

However, as the volume of the cell mixture received in the cell mixture holding portion 122, i.e. a surface area of the cell mixture holding portion 122, is increased, the angle of contact is decreased due to an increased effect of gravity.

Accordingly, in the embodiments of the present invention, an optimal cell separating environment for the easy cell separation is maintained by keeping a diameter of the cell mixture holding portion 122 not more than about 18 mm, preferably at a level of 14 mm such that the width of the cell mixture layer can be maintained within a range of 2 to 3 mm.

The lower plate support 123 is formed with a concave portion for accommodating the lower plate 121 therein such that the cell separation process can be performed in a state where the lower plate 121 is securely seated in the concave portion.

Further, the lower plate support 123 is installed to be movable within the lower housing 126 in a vertical direction. That is, a bolt-shaped connection is formed on a bottom surface of the lower plate support 123 to be engaged with a nut-shaped connection formed on the lower plate support moving dial 124 such that the lower plate support and thus the lower plate 121 can be moved vertically by means of the rotation of the dial 124.

The lower plate support moving dial 124 is installed such the lower plate support 123 can be moved vertically by rotating an exposed portion thereof in a horizontal direction in a state where a portion of the dial is exposed to the outside from a front side of the lower housing 126.

As the lower plate support moving dial 124 is moved horizontally, the lower plate support 123 is moved vertically to allow a vertical position of the lower plate 121 to be adjusted. Accordingly, the cell mixture received in the cell mixture holding portion 122 is adsorbed into the cell mixture adsorbing portion 112 to either create a cell mixture layer between the upper and lower plates 111 and 121 or remove the created cell mixture layer.

Here, in order to facilitate the cell separation process, a position or range where the cell mixture layer is created or removed may be indicated on the lower plate support moving dial 124 using letters or colors.

For example, numerals ‘1’, ‘2’ and ‘3’ are indicated on an edge of the lower plate support moving dial 124 in a counterclockwise direction in such a manner that the numeral ‘1’ means a state where the cell mixture layer is compressed, ‘2’ means a state where the cell mixture layer is maintained at a proper width, and ‘3’ means a state where the cell mixture layer has been removed.

In the cell separation apparatus 10 according to an embodiment of the present invention, the width between the upper and lower plates 111 and 121 can be set 2 mm for the ‘1’ state, 2.5 to 3 mm for the ‘2’ state, and 5 to 6 mm for the ‘3’ state when the diameter id the cell mixture holding portion 122 is 14 mm.

Accordingly, in the cell separation apparatus 10 of the embodiment of the present invention, both the lower plate support 123 and the lower plate support moving dial 124 perform a function as a means for adjusting the gap between the upper and lower plates 111 and 121 by moving the lower plate 121 to execute a process of creating a cell mixture layer and adjusting the width of or separating the created cell mixture layer.

In a case where a cell mixture layer is created or the created layer is divided to separate the cells while the gap between the upper and lower plates 111 and 121 is adjusted to be increased or decreased, the cell separation can be smoothly obtained without any additional equipment since the cell distribution due to the magnetic field and gravity in the layer and the creation or separation of the layer can be simultaneously determined.

Further, the dial stopper 125 is installed in the form of a protrusion at the ‘1’ and ‘3’ positions on the bottom surface of the lower plate support moving dial 124.

In addition, the dial stopper 125′ formed on the floor surface of the lower housing 126 is installed in the form of a protrusion at a position such that it is brought into contact with the ‘3’-position dial stopper 125 and thus not further rotated when the numeral ‘1’ is exposed to the outside from the front side of the lower housing 126 and that it is brought into contact with the ‘1’-position dial stopper 125 and thus not further rotated when the numeral ‘3’ is exposed to the outside.

Therefore, the lower plate support moving dial 124 is rotated only within the ‘1’ and ‘3’ positions by the dial stoppers 125 and 125′ such that a process of creating and separating the cell mixture layer can be conveniently performed without paying specific attention thereto.

The lower housing 126 is integrally coupled with the upper housing 115 in such a manner that the open top of the lower housing is connected to the open bottom of the upper housing. Some protrusions are formed around an outer rim of the lower housing 126 such that the lower housing can be easily coupled with the upper housing 115.

When the lower and upper housings 126 and 115 are coupled with each other, a closed space is defined between the housings such that an optimal environment in which cells are alive is maintained while moisture evaporation or temperature change in the cell mixture layer is minimized.

Furthermore, in a case where the cells other than the specific cells are tagged to the separated specific cells within the cell separation apparatus 10 according to the embodiment of the present invention, the purity of the specific cells can be further increased by separating and eliminating the other cells from the separated cells during the homogenization process in a state where the cell mixture layer is maintained.

A further dial stopper 125″ is installed at a front side of the lower plate 126 such that the lower plate support moving dial 124 can be rotated only within a limit where the layer is maintained when the homogenization process is performed.

The dial stopper 125″ can be installed to move leftward or rightward. Thus, a portion of the dial stopper protruding toward the lower plate support moving dial 124 is also moved leftward or rightward within the lower housing 126.

Therefore, if the dial stopper 125″ is moved leftward, the ‘1’-position dial stopper 125 is hindered by the protruding portion of the dial stopper 125″ from being further rotated when the dial stopper 125 is positioned such that the numeral ‘2’ is exposed to the outside. Thus, the homogenization process can be rapidly and conveniently performed while the cell mixture layer is maintained.

However, if the dial stopper 125″ is moved rightward, the ‘1’-position dial stopper 125 is not hindered by the protruding portion of the dial stopper 125″ from being rotated and the lower plate support moving dial 124 can thus be turned up to the ‘3’ position in a counterclockwise direction even when the dial stopper 125 is positioned such that the numeral ‘2’ is exposed to the outside.

Considering that the upper and lower plates 111 and 121 are newly exchanged whenever cells are separated, the cell separation apparatus 10 of the embodiment of the present invention may be divided into a cell separation chip composed of the upper and lower plates 111 and 121, and a cell separation executing unit for adjusting the gap between the upper and lower plates 111 and 121 and executing the cell operation by means of the magnetic field applied between the plates.

A process of separating cells from a cell mixture by using the cell separation apparatus according to the embodiment of the present invention will be described.

As shown in FIG. 3, the upper body 11 of the cell separation apparatus 10 of the present invention is first disconnected from the lower body 12. Then, a cell mixture in which the specific cells tagged with magnetic beads are mixed is injected and received in the lower plate 121 of the lower body 12, i.e. each cell mixture holding portion 122 with a diameter of 14 mm, at an amount of 500 [symbol] .

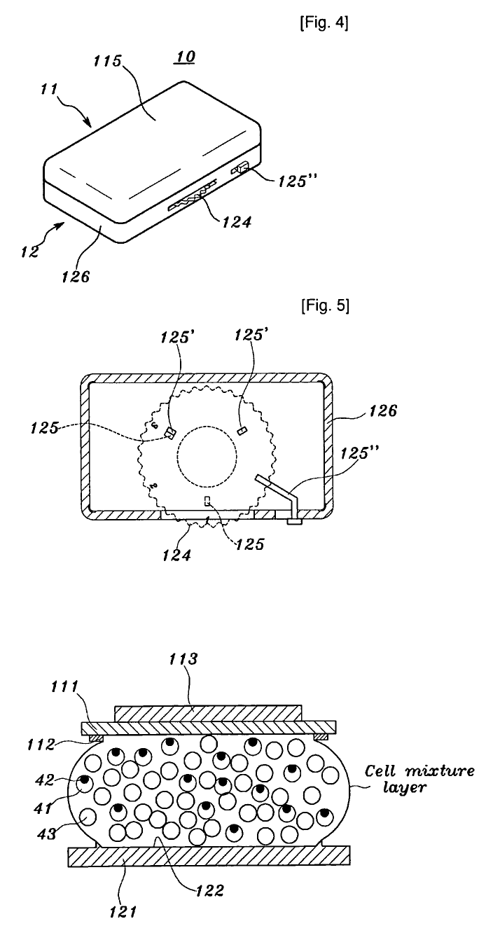

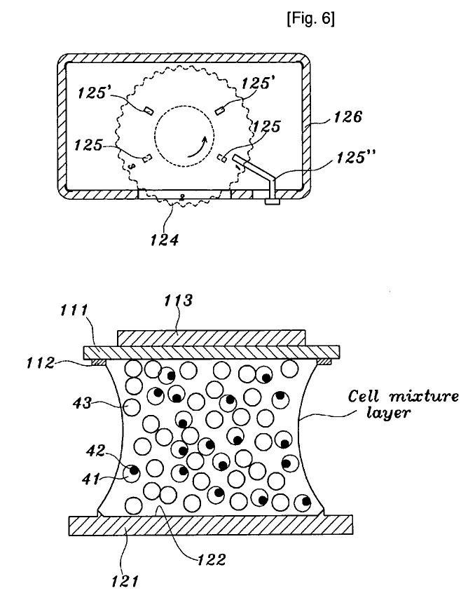

As shown in FIG. 4, the upper body 11 is covered onto the lower body 12 such that they are integrally coupled with each other. Here, in a case where the numeral ‘1’ indicated on the lower plate support moving dial 124 of the lower body 12 is positioned to be exposed to the outside as shown in FIG. 5, the ‘3’-position dial stopper 125 is brought into contact with the dial stopper 125′ installed on the floor surface of the lower housing 126, and thus, the lower plate support moving dial 124 cannot be further rotated.

In such a case, the cell mixture layer in which the specific cells and the other cells are uniformly distributed between the cell mixture adsorbing portion 112 of the upper plate 111 and the cell mixture holding portion 122 of the lower plate 121 in a compressed state.

Next, if the lower plate support moving dial 124 positioned with the numeral ‘1’ exposed to the outside is rotated in a counterclockwise direction to cause the numeral ‘2’ to be exposed to the outside, the dial stopper 125 installed on the bottom surface of the lower plate support moving dial 124 is moved rightward but is not brought into contact with the dial stopper 125′ installed on the floor surface of the lower housing 126.

In such a case, the lower plate 121 is moved downward to thereby increase the gap between the lower and upper plates such that the compressed cell mixture layer can be formed into a layer with a predetermined width suitable for the cell separation.

However, it is not always preferred that the thickness of the cell mixture layer created between the upper and lower plates 111 and 121 be kept constant whenever cells are separated.

In general, as the vertical width of the cell mixture layer, i.e. the gap between the upper and lower plates 111 and 121 is increased, the cell separation can be clearly performed by means of the magnetic field applied to the specific cells and the gravity applied to the other cells.

However, the compatibility (generality) that all the characteristics (size, density, degree of magnetization and the like) of the magnetic beads varying according to the manufacturers can be accepted after the cell mixture layer has been created should be obtained, or the characteristics of specific cells themselves should be properly controlled since the degrees of the specific cells contained in the cell mixture (cell concentration) are different from each other. Further, an adsorption degree may vary according to whether the material of the upper and lower plate 111 and 121 as a cell separation chip is hydrophilic or hydrophobic. Thus, the gap between the upper and lower plates 111 and 121 should be adjusted to maintain the cell separation in an optimal state.

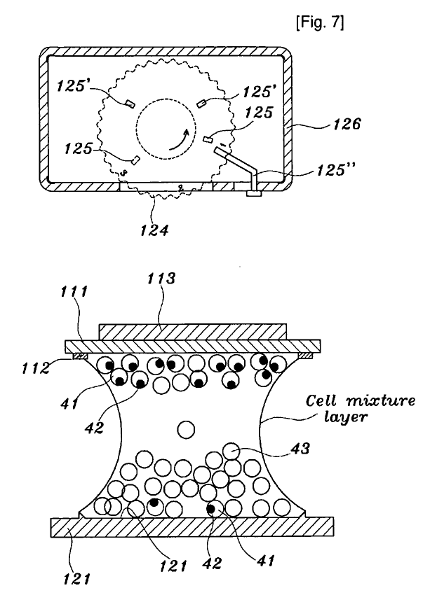

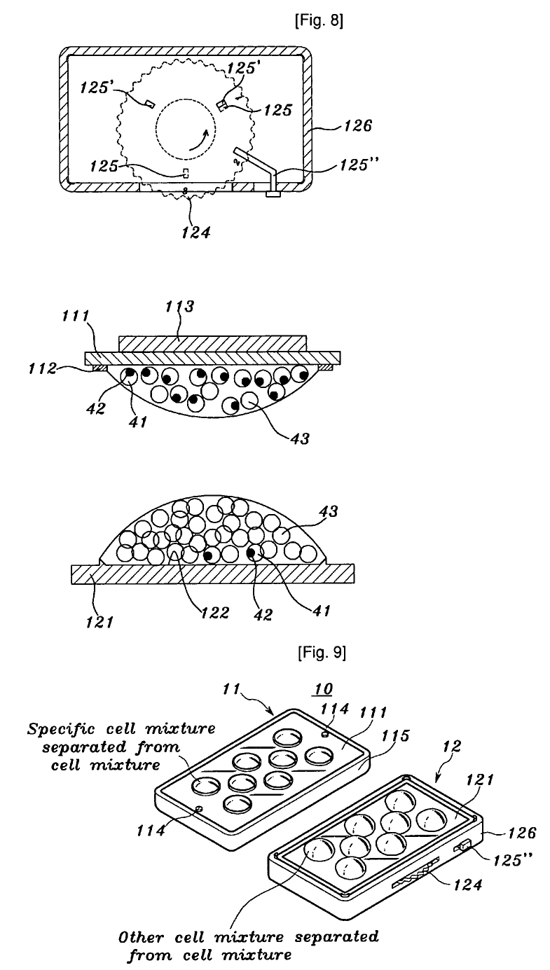

Here, if it is assumed that the optimal cell separation state can be maintained by slightly increasing the width of the cell mixture layer so created, the lower plate support moving dial 124 is slightly further rotated in a counterclockwise direction as compared with when the numeral ‘2’ is exposed to the outside as shown in FIG. 7. That is, the lower plate 121 can be moved downward slightly further than as shown in FIG. 6.

In such a case, the dial stopper 125 installed on the bottom surface of the lower plate support moving dial 124 is further moved in a counterclockwise direction but is not yet brought into contact with the dial stopper 125′ installed on the floor surface of the lower housing 126.

Then, if the current state is maintained for 7 to 15 minutes while the magnetic field is applied to the cell mixture layer with an optimal width through the magnets 113 disposed on the top surface of the upper plate 111, the specific cells in the cell mixture layer are moved toward the upper plate 111. At the same time, the other cells in the cell mixture layer are also moved toward the lower plate 121 by means of gravity.

Finally, if the lower plate support moving dial 124 is turned in a counterclockwise direction with the numeral ‘3’ exposed to the outside, the ‘1’-position dial stopper 125 is also moved in the counterclockwise direction and is brought into contact with the relevant dial stopper 125′ installed on the floor surface of the lower housing 126. Thus, it is possible to prevent the lower plate support moving dial 124 from being further rotated.

In such a case, the lower plate 121 will be completely moved downward such that the cell mixture layer can be separated. As a result, the specific cells and the other cells are divided and then positioned in the upper and lower plates 111 and 121, respectively.

Therefore, the dial stopper 125 and 125′ perform the functions of creating the cell mixture layer, adjusting the width of the created cell mixture layer, separating the layer and separating the cells in a convenient way while being turned within the range of the ‘1’ and ‘3’ positions.

As shown in FIG. 9, the specific cells and the other cells are divided and then received in the upper plate 111 of the upper body 11 and the lower plate 121 of the lower body 12, respectively. Thereafter, necessary cells are collected and then utilized.

As shown in FIG. 4, when the upper body 11 is covered onto the lower body 12 such that they are integrally coupled with each other, the lower plate support moving dial 124 of the lower body 12 is positioned in a state where the numeral ‘2’ or ‘3’ is exposed to the outside. Preferably, the lower plate support moving dial 124 is turned in a clockwise or counterclockwise direction to maintain or create the cell mixture layer.

According to the present invention, the cell separation apparatus 10 can be simply operated and be portable due to the small size thereof. Further, specific cells that should be maintained at a temperature of 4° C. can be simply stored and then separated in a refrigerator without moving the cell separation apparatus containing the specific cells to a specific place or using additional cooling equipment.

The cells adsorbed to the upper plate 111 should become the specific cells. In fact, however, the other cells may be adsorbed to the upper plate 111 when the cell mixture layer is created. Alternatively, a portion of the other cells adhering to the specific cells may be attracted together with the specific cells when they are attracted toward the upper plate 111 by means of the magnetic field generated from the magnets 113. Therefore, unnecessary cells other than the specific cells adsorbed to the upper plate 111 should also be separated.

Accordingly, in order to create an environment in which the other cells adhering to the specific cells can be optimally separated, the separated other cells are removed from the lower plate 121 or the used lower plate is replaced with a new lower plate.

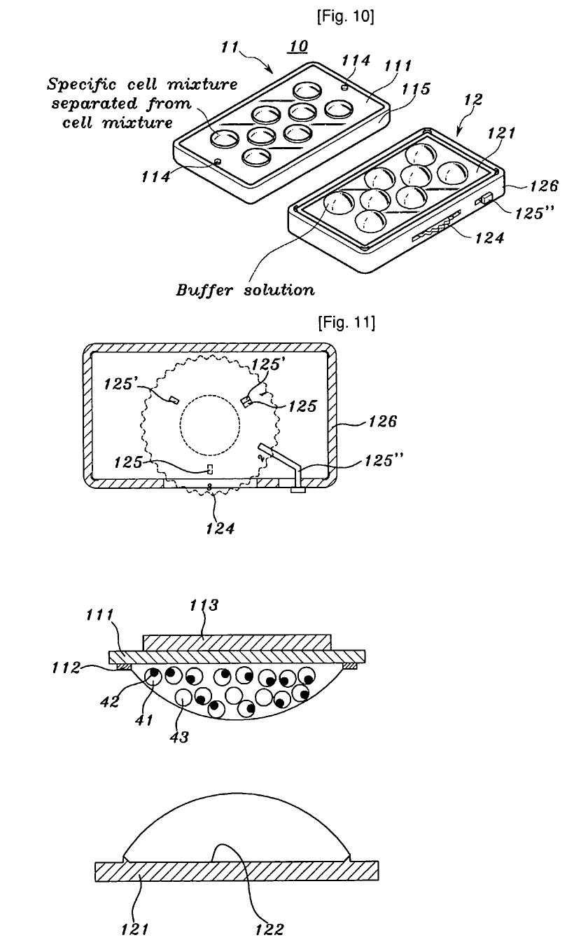

Then, a buffer solution containing no cells is injected and accommodated in the lower plate 121 as shown in FIG. 10. Finally, the upper body 11 having the upper plate 111 with the specific cells adsorbed therein is covered onto the lower body 12 having the lower plate 121 such that the two bodies are integrally coupled with each other.

As shown in FIG. 11, since the lower plate support moving dial 124 is positioned such that the numeral ‘3’ can be exposed to the outside, the specific cells are positioned in the upper plate 111 and the buffer solution is positioned in the lower plate 121, but a cell mixture layer is not yet created between the two plates.

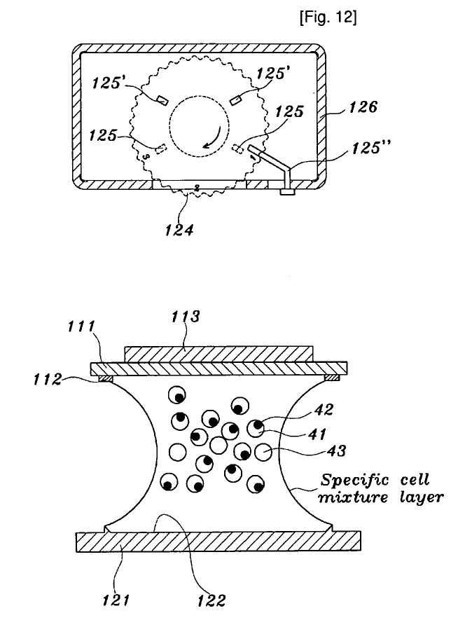

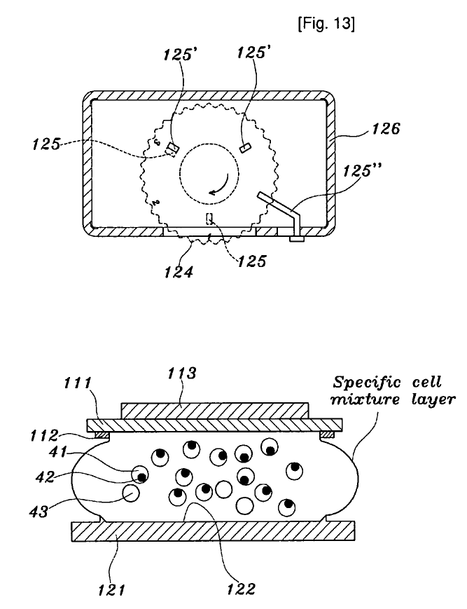

Next, as shown in FIGS. 12 and 13, if the lower plate support moving dial 124 positioned at the ‘3’ position is sequentially turned to the ‘2’ and ‘1’ positions in a clockwise direction, the dial stopper 125 installed on the bottom surface of the lower plate support moving dial 124 is moved in a clockwise direction and then brought into contact with the dial stopper 125′ installed on the floor surface of the lower housing 126 such that the lower plate support moving dial 124 cannot be further turned in a clockwise direction.

In such a case, as the lower plate 121 is moved upward, the gap between the upper and lower plates is decreased. Thus, the specific cells adsorbed in the upper plate 111 and the buffer solution contained in the lower plate 121 are mixed to create a specific cell mixture layer suitable for the cell separation such that the created layer is again in a vertically compressed state.

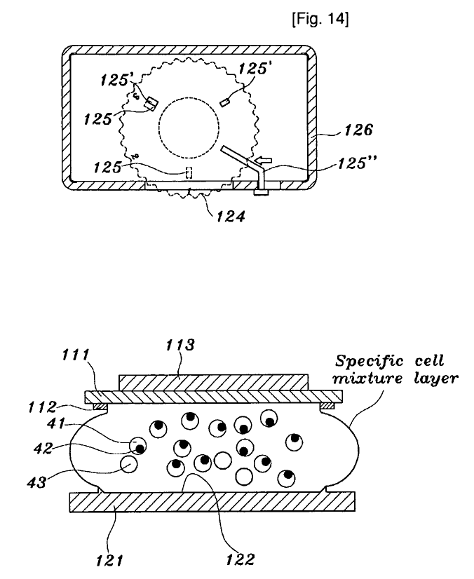

Further, as shown in FIG. 14, while the lower plate support moving dial 124 is still maintained at the ‘1’ position, the dial stopper 125″ is moved leftward.

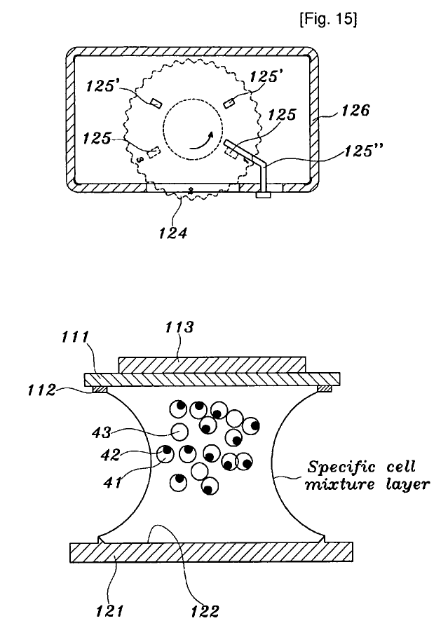

Furthermore, as shown in FIG. 15, if the lower plate support moving dial 124 positioned at the ‘1’ position is turned in a counterclockwise direction to be positioned at the ‘2’ position after the dial stopper 125″ has been moved leftward, the dial stopper 125″ prevents the dial stopper 125 positioned at the ‘1’ position from being rotated in a counterclockwise direction such that the lower plate support moving dial 124 cannot be further turned in the counterclockwise direction.

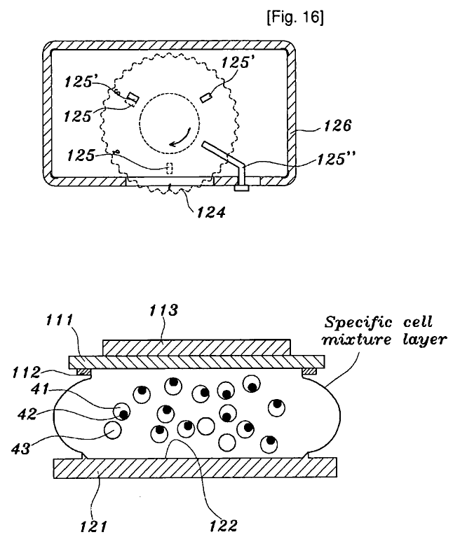

Next, the processes illustrated in FIGS. 15 and 16 are repeated several times, e.g. 10 times, while maintaining the specific cell mixture layer created between the upper and lower plates 111 and 121 to rapidly and conveniently homogenize the specific cell mixture layer.

Then, the specific cell mixture layer is shaken vertically to the utmost in a state where the specific cell mixture layer is maintained, and consequently, the other cells that adhere to the upper plate 111 together with the specific cells reacting with the magnets 113 are separated such that the layer can be in a single cell state.

After the homogenization process has been completed, the specific cell mixture layer is left alone for 7 to 15 minutes in a state where the numeral ‘2’ is exposed to the outside, as shown in FIG. 15. Then, the specific cells in the homogenized cell mixture layer are moved toward the upper plate 111 by means of the magnetic field applied thereto through the magnets 113, whereas the other cells in the cell mixture layer are moved toward the lower plate 121 by means of gravity.

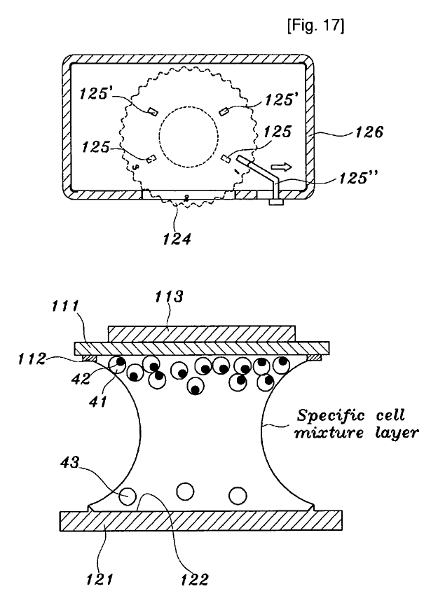

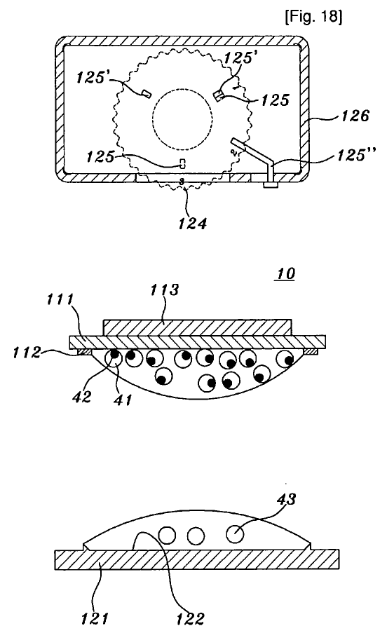

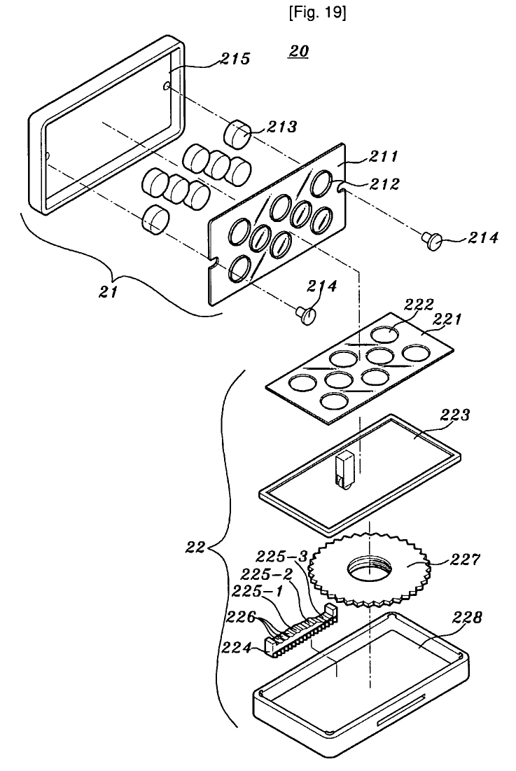

If the dial stopper 125″ is moved rightward and the lower plate support moving dial 124 is then turned in the counterclockwise direction toward the ‘3’ position in such a state as shown in FIG. 17, the ‘1’-position dial stopper 125 is moved rightward and brought into contact with the dial stopper 125′ installed on the floor surface of the lower housing 126 to restrict the rotation of the lower plate support moving dial 124.

In such a case, the lower plate 121 has completely will be completely moved downward such that the cell mixture layer can be separated. As a result, the specific cells and the other cells are divided and then positioned in the upper and lower plates 111 and 121, respectively.

That is, the other cells contained in the separated specific cells are additionally separated and removed from the upper plate 111 to thereby markedly enhance the purity of the necessary specific cells.

FIG. 19 shows a cell separation apparatus 20 according to another embodiment of the present invention. The cell separation apparatus shown in FIG. 19 is identical to the cell separation apparatus 10 of the previous embodiment except their gap adjusting means for adjusting the gap between the upper and lower plates. Hereinafter, the gap adjusting means for adjusting the gap between the upper and lower plates will be mainly described.

In the cell separation apparatus 10 according to the previous embodiment of the present invention, the lower plate support moving dial 124 which serves as a gap adjusting means for adjusting the gap between the upper and lower plates and includes the nut-shaped connection engaged with the bolt-shaped connection formed on the bottom surface of the lower plate support 123 is rotated to vertically move the lower plate support 123 such that the vertical position of the lower plate 121 can be adjusted. On the other hand, in the cell separation apparatus 20 according to this embodiment of the present invention, a bar moving dial 227 having a pinion gear portion meshed with a rack portion formed on a side surface of a lower plate support moving bar 224 is rotated to horizontally move the lower plate support moving bar 224 with three steps formed on an upper surface of the lower plate support moving bar 224 such that a lower plate support 223 can be moved vertically by using a roller formed on a bottom surface of the lower plate support 223.

The three steps in the cell separation apparatus 20 according to this embodiment of the present invention perform the same function as the ‘1’, ‘2’ and ‘3’ positions indicated on the edge of the lower plate support moving dial 124 of the cell separation apparatus 10 according to the previous embodiment of the present invention.

The lower plate support 223 includes a roller, unlike the bolt-shaped connection formed on the lower plate support 123 of the cell separation apparatus 10 according to the previous embodiment of the present invention.

The lower plate support moving bar 224 is formed with three steps whose levels are decreased from left to right to implement the vertical motion of the lower plate support 223 in correspondence with the ‘1’ to ‘3’ positions. However, the number of steps may be changed, or the steps may be connected in an inclined manner.

The roller formed below the lower plate support 223 is kept in contact with any one of the three steps formed on the upper surface of the lower plate support moving bar 224.

Therefore, if the lower plate support moving bar 224 is moved in a horizontal direction along its longitudinal axis, the position of the steps brought into contact with the roller is changed to vertically move the lower plate support 223 including the roller.

Grooves 225 for temporarily restricting the motion of the lower plate support 223 are formed on the three steps, respectively.

The grooves 225 cause the roller to be temporarily held therein until a pre-determined force is applied to the roller. Thus, the aforementioned positions ‘1’, ‘2’ and ‘3’ can be sensibly divided such that a user can easily create the layer, adjust the width of the created layer and separate the layer.

Further, a lower plate support shaking portion 226 extending from the groove 225 and formed with a plurality of grooves is installed on the upper surface of the lower plate support moving bar 224.

The vibration generated from the lower plate support shaking portion 226 is transmitted to the lower plate 221 through the lower plate support 223 to perform a process of homogenizing a cell mixture layer containing cells other than the specific cells.

The bar moving dial 227 is configured in such a manner that a portion of its edge is exposed to the outside from a front side of a lower housing 228. If the exposed portion of the bar moving dial is turned in a counterclockwise direction, the lower plate support moving bar 224 is moved from right to left.

At this time, the pinion gear portion of the bar moving dial 227 is threadedly engaged with the rack portion of the lower plate support moving bar 224. That is, as the bar moving dial 227 is turned, the lower plate support moving bar 224 is moved horizontally. Thus, the level of the steps brought into contact with the roller of the lower plate support 223 is changed to allow the lower plate support 223 to be vertically moved.

A process of separating cells using the cell separation apparatus 20 according to this embodiment of the present invention so configured will be explained as follows.

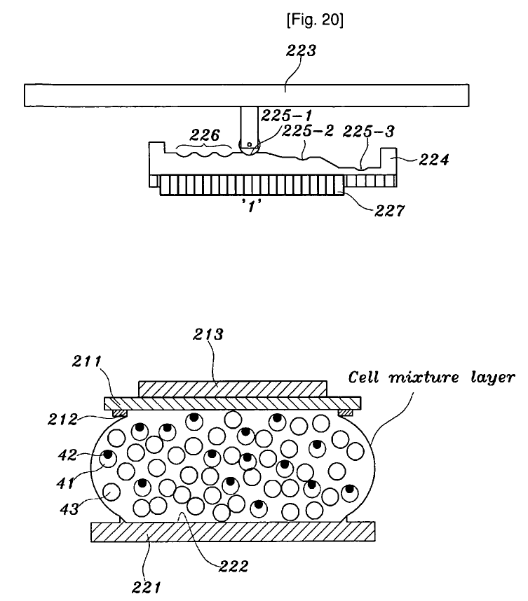

A cell mixture is first injected and received in a lower body 22 of the cell separation apparatus according to this embodiment of the present invention, and an upper body 21 is then covered onto the lower body 22 such that the two bodies can be integrally coupled with each other. As shown in FIG. 20, if the bar moving dial 227 is positioned such that the numeral ‘1’ is exposed to the outside, the roller of the lower plate support 223 is temporarily fixed and positioned in the first groove 225-1 corresponding to the ‘1’ position.

In such a case, a cell mixture layer in which the specific cells and the other cells are uniformly distributed is created in a compressed state between a cell mixture adsorbing portion 212 of an upper plate 211 and a cell mixture holding portion 222 of the lower plate 221.

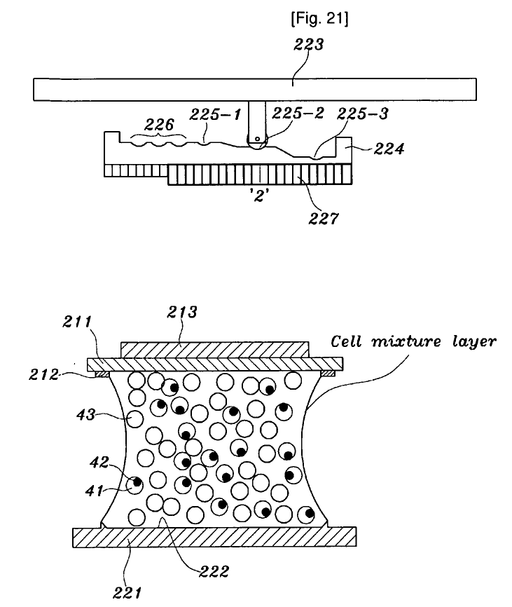

Next, if the bar moving dial 227 is turned in the counterclockwise direction from the ‘1’ position to another position where the numeral ‘2’ is exposed to the outside, the lower plate support moving bar 224 is moved leftward to thereby move the lower plate support 223 downward and to increase a gap between the upper and lower plates as shown in FIG. 21. Therefore, the cell mixture layer with a width suitable for the cell separation can be created.

In such a case, the roller of the lower plate support 223 is kept in a state where it is temporarily fixed into the second groove 225-2 corresponding to the ‘2’ position.

Then, if the current state is maintained for 7 to 15 minutes while the magnetic field is applied to the cell mixture layer with an optimal width through the magnets 213 disposed on the top surface of the upper plate 211, the specific cells in the cell mixture layer are moved toward the upper plate 211. At the same time, the other cells in the cell mixture layer are also moved toward the lower plate 221 by means of gravity.

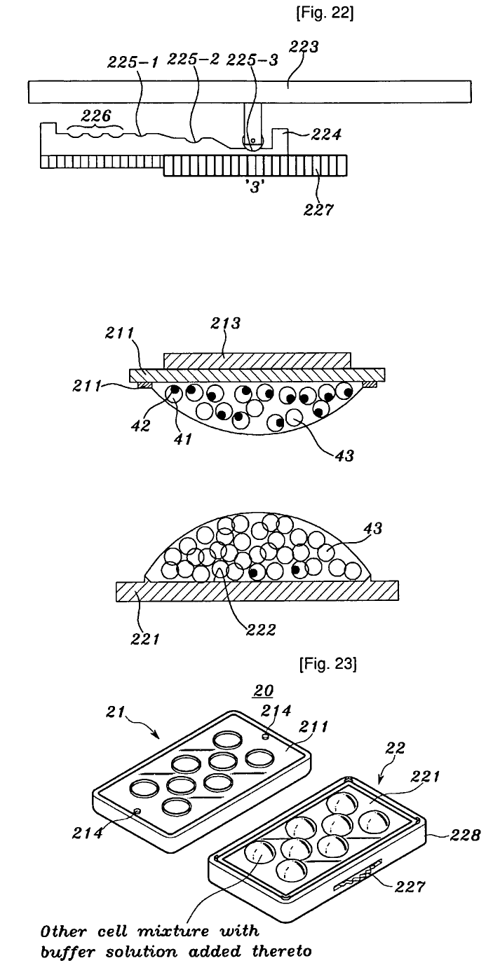

Finally, if the bar moving dial 227 is turned in the counterclockwise direction from the ‘2’ position to another position where the numeral ‘3’ is exposed to the outside, the lower plate support moving bar 224 is moved leftward to thereby to completely move the lower plate support 223 downward and to increase the gap between the upper and lower plates, as shown in FIG. 22. Thus, the cell mixture layer is separated such that the specific cells and the other cells are divided and then positioned in the upper and lower plates 211 and 221, respectively.

In such a case, the roller of the lower plate support 223 is kept in a state where it is temporarily fixed into the third groove 225-3 corresponding to the ‘3’ position.

Accordingly, as shown in FIG. 9, since the specific cells and the other cells are divided and then positioned in the upper and lower plates 211 and 221, respectively, necessary cells will be collected and utilized later.

In the cell separation apparatus 20 according to this embodiment of the present invention, the steps of creating the cell mixture layer, adjusting the thickness of the later and separating the layer can be sensibly divided at temporarily fixed conditions such that a user can feel the conditions and perform the convenient and precise cell separation process.

The cells adsorbed to the lower plate 221 should become the cells other than the specific cells. In fact, however, a portion of the specific cells adhering to the other cells may be attracted together with the other cells when they are attracted toward the lower plate 221 by means of gravity.

Here, the cells to be collected generally become the specific cells tagged with magnetic beads. However, if they are clinically utilized, the other cells with no magnetic beads tagged thereto may be employed.

Accordingly, the cells other than unnecessary specific cells adsorbing to the upper plate 211 should be separated.

In order to create an environment where the specific cells adhering to the other cells can be optimally separated, the separated specific cells should be removed from the lower plate 121 or the used lower plate is replaced with a new lower plate as shown in FIG. 23.

Further, the upper body 21 including the empty upper plate 211 is covered onto the lower body 22 including the lower plate 221 injected with a buffer solution with no cells contained therein at an amount corresponding to a degree that the specific cells transferred to the upper plate 211 are replenished, such that the upper and lower bodies can be integrally coupled with each other.

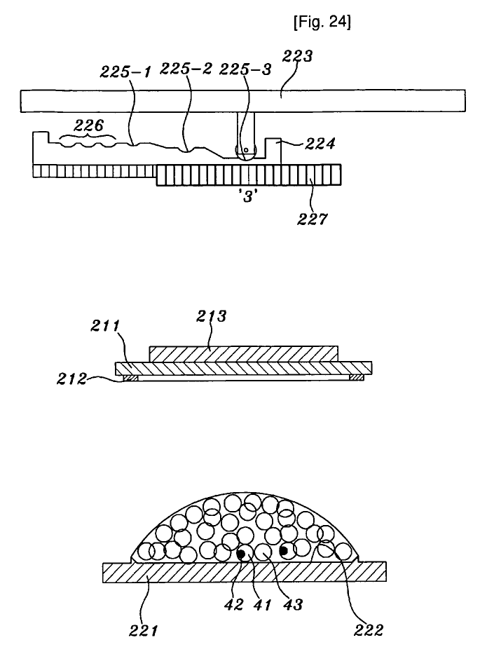

In such a case, as shown in FIG. 24, the bar moving dial 227 is positioned in a state where the numeral ‘3’ is exposed to the outside. That is, no cell mixture layer is created in a state where the upper plate 211 is only empty.

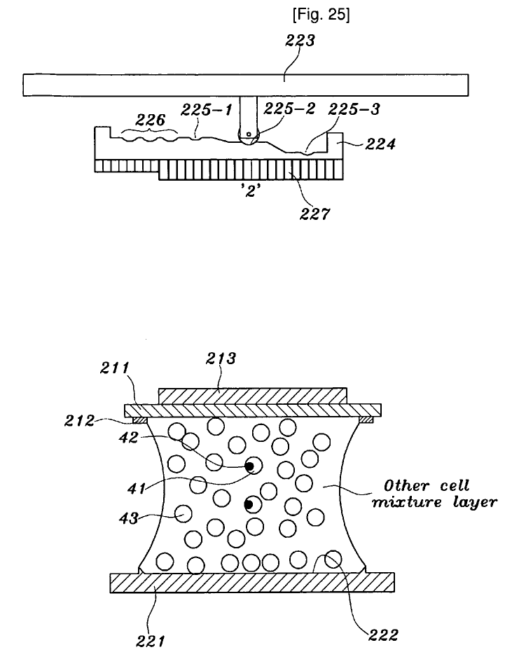

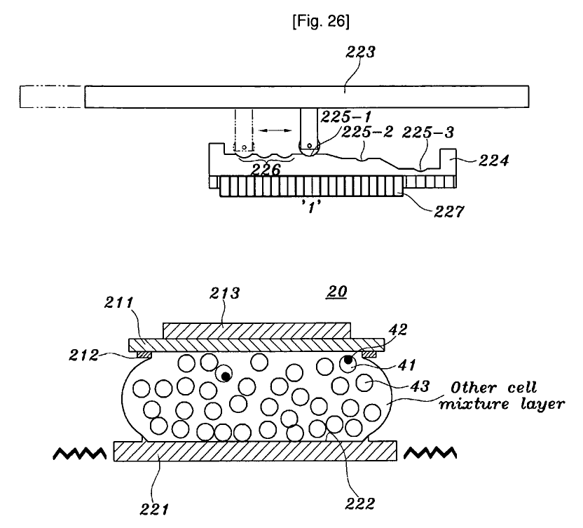

As shown in FIGS. 25 and 26, if the bar moving dial 227 is turned in a clockwise direction from the ‘3’ position through the ‘2’ position to the ‘1’ position, the lower plate support moving bar 224 is moved rightward and the roller of the lower plate support 223 is positioned on the highest step. Thus, the lower plate support 223 can be pushed upward.

In such a case, as the lower plate 221 is moved upward, the gap between the upper and lower plates is decreased such that the cell mixture of the other cells in the lower plate 221 absorbs against the upper plate 211 to create the mixture layer of the other cells suitable for the cell separation. Then, the created layer is in a compressed state.

Next, as shown in FIG. 26, if the bar moving dial 227 is further turned from the ‘1’ position in the clockwise direction (the lower plate support 223 is moved at a dotted line of FIG. 26), the vibration generated when the roller of the lower plate support 223 travels rightward along the lower plate support shaking portion 226 in a state where they are brought into contact with each other is transmitted to the lower plate 221 through the lower plate support 223.

At this time, even when the bar moving dial 227 is turned in the counterclockwise direction to move the roller of the lower plate support 223 rightward up to the first groove 225-1 corresponding to the ‘1’ position, the vibration generated when the roller of the lower plate support 223 travels leftward along the lower plate support shaking portion 226 in a state where they are brought into contact with each other is transmitted to the lower plate 221 through the lower plate support 223.

The homogenization process is performed while the vibration transmitted to the lower plate 221 is applied to the other cell mixture layer. This process is repeated several times, e.g. less than 10 times, while maintaining the other cell mixture layer created between the upper and lower plates 211 and 221.

Then, since a maximum vibration can be applied to the maintained other cell mixture layer, the specific cells adhering to lower plate 221 together with the other cells fallen onto the lower plate 221 by means of gravity come off from the lower plate 221. Subsequently, the specific cells are moved toward the upper plate 211 by means of the magnetic field applied thereto from the magnets 213.

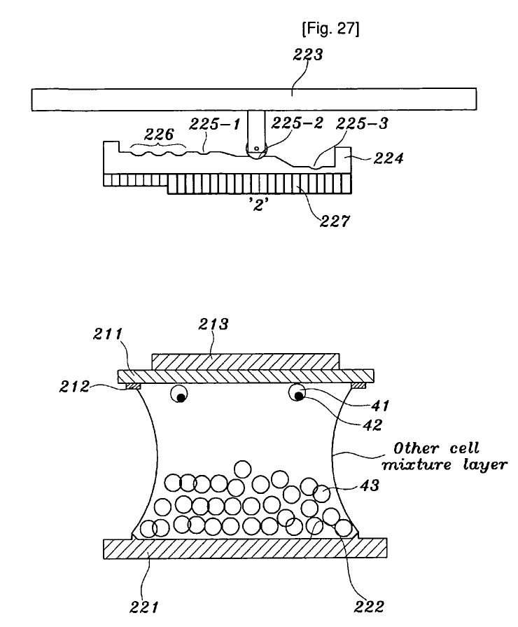

After the homogenization process has been performed, the cell mixture layer is left alone for 7 to 15 minutes in a state where the roller is temporarily fixed in the second groove 225-2 by positioning the bar moving dial at the ‘2’ position, as shown in FIG. 27. As a result, the specific cells are moved toward the upper plate 211 by means of the magnetic field applied thereto from the magnets 213 while the other cells are moved toward lower plate 221 by means of gravity.

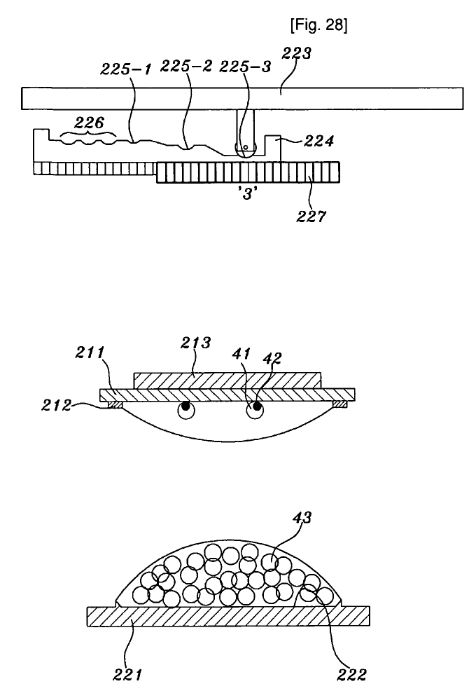

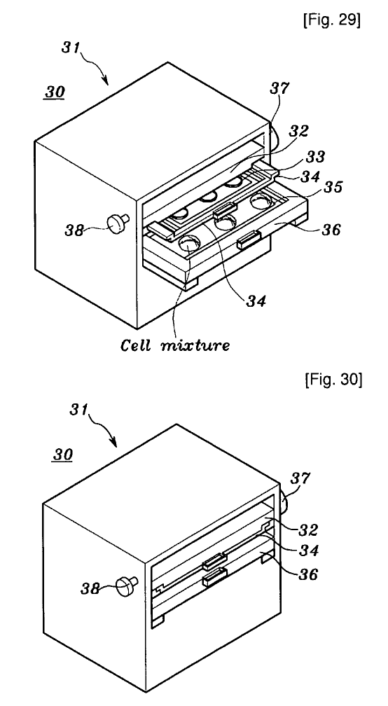

In such a state, if the bar moving dial 227 is further turned in the counterclockwise direction from the ‘2’ position to the ‘3’ position, the lower plate support moving bar 224 is moved leftward and thus the lower plate support 223 is completely moved downward as shown in FIG. 28. Therefore, the gap between the upper and lower plates are increased to thereby separate the cell mixture layer such that the specific cells and the other cells are divided and then received in the upper and lower plates 211 and 221, respectively.

At this time, the roller of the lower plate support 223 is kept in a state where it is temporarily fixed in the third groove 225-3 for restricting the motion of the lower plate support.

Accordingly, the unnecessary specific cells can be additionally separated from the other cell mixture from which the specific cells have been previously separated and finally removed from the lower plate 221. Thus, the purity of the other cells can be greatly enhanced.

A cell separation apparatus 30 according to a further embodiment of the present invention is identical to those of the previous embodiments of the present invention except the following points. That is, in the previous embodiments of the present invention, the upper body to which the magnetic field is applied is covered onto the lower body in which the cell mixture is contained such that the two bodies are integrally coupled with each other. At this state, the lower plate is moved to adjust the gap between the upper and lower plates such that the specific and other cells are separated. On the other hand, in this embodiment of the present invention, the upper and lower plates are installed in a single housing and the upper plate is then moved to adjust the gap between the upper and lower plates such that the specific and other cells are separated. Hereinafter, the foregoing difference will be mainly described.

In the cell separation apparatus 30 according to the further embodiment of the present invention, an upper plate support 34 and a lower plate support 36 are installed to be movable in a fore and aft direction within a housing 31 with a portion thereof opened forward. Here, the lower plate support 36 is fixed installed at a lower portion of the housing 31, whereas the upper plate support 34 is positioned above the lower plate support 36 by a certain distance and installed to be vertically movable within the housing 31.

The upper plate support 34 and the lower plate support 36 are formed with recesses in which an upper plate 33 and a lower plate 35 are received, respectively. That is, the upper and lower plates 33 and 35 are installed within the recesses, respectively, such that cell mixture adsorbing portions and cell mixture holding portions face each other.

Further, a magnet 32 is installed above the upper plate support 34 to face a top surface of the upper plate 33.

In addition, the upper plate support 34 is threadedly engaged (not shown) with an upper plate support moving dial 37 installed on an outer side of the housing 31 such that it can be vertically moved by means of the rotation of the upper plate support moving dial 37.

Here, when the upper plate support 34 is vertically moved, both the magnet 32 and the upper plate 33 are moved together with the support 34.

Further, a dial stopper 38 is installed on another outer side of the housing 31 to restrict the vertical motion of the upper plate support 34 which is vertically moved as the upper plate support moving dial 37 is turned or rotated.

The dial stopper 38 restricts the upward motion of the upper plate support 34 by using a protrusion formed to protrude into the housing 31 when the dial stopper is pushed, such that a cell mixture layer can be maintained.

A process of separating cells using the cell separation apparatus according to the further embodiment of the present invention so configured will be hereinafter described.

First, the lower plate support 36 is taken out in a front loading mode toward a user standing in front of the cell separation apparatus and the cell mixture is then injected and contained in the lower plate 35 in a state where the lower plate 35 is securely seated in the recess of the lower body with the cell mixture holding portions facing upward. Then, the lower plate support 36 is again pushed rearward into the housing 31.

Next, the upper plate support 34 is taken out in a front loading mode toward the user standing in front of the cell separation apparatus and the upper plate 33 is securely seated in the recess of the upper body with the cell mixture adsorbing portions facing upward. The aforementioned processes may be performed in a reverse order.

Then, the magnet 32, the upper plate 33 and the upper plate support 34 are vertically moved together with one another as the upper plate support moving dial 37 is turned. Thus, the upper plate 33 is moved toward the lower plate 35 to be closer to each other to create the cell mixture layer. Then, the magnetic field is applied to the created cell mixture layer from the upper plate 33 using the magnet 32 to move the specific cells and the other cells toward the upper plate 33 and the lower plate 35, respectively. Finally, the upper plate 33 is again moved far away from the lower plate 35 to allow the cell mixture layer to be separated.

Here, if the upper plate support 34 and the lower plate support 36 are brought into contact with each other, the cell mixture layer can be created in a compressed state between the upper and lower plates 33 and 35.

Finally, the specific cells adsorbed in the upper plate 33 can be recovered after the upper plate support 34 is pulled out, or the other cells in the lower plate 35 can be recovered after the lower plate support 36 is pulled out.

Further, the homogenization process performed in the cell separation apparatus 10 and 20 according to the previous embodiments of the present invention may be executed by operating the dial stopper 38.

The process of separating cells from a cell mixture according to the embodiments of the present invention was tested as follows.

A cell mixture obtained by flushing bone marrow cells is first labeled with magnetic beads. The concentration of the cell mixture used to separate specific cells from the cell mixture for each antibody is 10⁷/ [symbol] , and the cell separation process is performed in a cold chamber at a temperature below 4° C. to exclude the temperature effects.

In case of the cell separation apparatus 10 and 20 according to the embodiments of the present invention, since the upper and lower bodies are coupled integrally with each other, a closed space is created and the cell mixture is injected in the cell mixture holding portions. Then, if the cell separation apparatus including the cell mixture is placed for 7 to 15 minutes within a refrigerator or the like of which inside temperature is kept at about 4° C. in a state where the lower plate moving dial is placed at the ‘3’ position, the cell separation process is fully completed without any additional operations or cold chamber. Thus, it is very convenient to perform the cell separation process.

When it is defined that the separated specific cells are positive while the other cells are Negatives, the results of the separation efficiency of positive obtained using a variety of antibodies can be expressed as follows.

TABLE 1

| | Content of Specific | |
|---|---|---|
| Antibody | Cells Contained in | Separation |
| Used | Control | Efficiency |
| CD45 | 80% | 97.5% |
| Ter119 | 13.3% | 98% |
| Sca1 | 2.09% | 28% |
| SSEA1 | <1% | 19.5% |
| SSEA3 | <1% | 13% |

Further, after the cells other than the separated specific cells, i.e. negative, have been collected, a separation error ratio (a ratio that the positive remains in the negative in a state where the positive is not fully separated) is confirmed. The results are expressed as follows.

TABLE 2

| | Content of Specific | |
|---|---|---|
| Antibody | Cells Contained in | Separation |
| Used | Control | Efficiency |
| CD45 | 80% | 5.4% |
| Ter119 | 13.3% | 18.3% |
| Sca1 | 2.09% | 1.8% |
| SSEA1 | <1% | 0.8% |
| SSEA3 | <1% | 0.5% |

The process of enhancing the purity of cells separated by applying a magnetic field to a cell mixture according to the embodiments of the present invention was specifically tested as follows.

In an experiment example of the present invention, the state of cells was measured from the negative solution collected after the cell separation process such that the error ratio can be confirmed by checking how much the positive is contained in the solution in the lower plate, i.e. the negative solution.

Basically, the cells used in the experiment example are cells reacting with the Ter119 antibody, the magnetic field from the magnet is 0.5 T, and the exposed period of time is 9 minutes.

As seen from Table 3, the separation efficiency when the specific cells exist (positive exists) in the cell mixture at a content level of 13.3% (Control.002 means a control and is used to compare the state of cells and the separation efficiency by measuring the state of cell mixture before the test) is expressed as follows.

As seen from Table 4, MB-P.002 corresponds to the measurement of the state of positive after the separation which indicates a separation efficiency of 93.77%. At the same time, MB1-N.023 shows that the positive is contained in the negative at a content level of 10.10%.

Here, the process of relatively enhancing the purity of the negative by removing the positive from the negative solution containing 10.10% of the positive was performed. It was also confirmed that an error ratio was decreased from an initial ratio (i.e., 10.10%) to 8.01% when examined under the assumption that the positive in the negative fraction is considered as an error.

Accordingly, it was confirmed that the positive contained in the negative solution as an error was decreased by about 20.7% and thus the purity of the negative was increased by about 2.3%.

In the case of the positive, cells reacting with the Sca1 antibody were tested under the same conditions (i.e., exposure to the magnetic field of 0.5 T for 9 minutes).

TABLE 5

| | | Positive | Negative |
|---|---|---|---|
| | | (Purity En- | (Positive |
| | Positive | hancement) | Error) |
| Control | 2.09 | | |
| Cell Separation | 6.30% | 13.55% | 2.51% |
| Apparatus according to | | | |
| Present Embodiments | | | |

As shown in Table 5, when the cell mixture (control) containing 2.09% of the positive are separated, it was confirmed that the content of the positive is 6.30% and the content of the negative is 2.51%.

Here, since the content of the specific cells is as low as 2.09%, the process of enhancing the purity of the positive was additionally performed. Thus, it was confirmed that the content of the positive was increased to 13.55% corresponding to about 115% increase as compared with the initial content.

The present invention is not limited to the aforementioned embodiments of the present invention. Various modifications and changes can be made thereto within the scope of the present invention defined in the appended claims. However, the changes and modifications should be construed as falling within the scope of the present invention.

Although it has been described in the embodiments of the present invention that the upper or lower plate is moved directly by the user to create the cell mixture layer, adjust the width of the created layer and separate the layer, a driving means such as a motor and a microprocessor for controlling the motor may be utilized to automatically operate the cell separation apparatus based on the predetermined algorithm instead of using the manual operation of the user.

### INDUSTRIAL APPLICABILITY

According to the cell separation apparatus and method of the present invention, the necessary cells can be separated through the process of creating the cell mixture layer, adjusting the thickness of the layer and separate the layer by adjusting the vertical gap between the upper and lower plates of the cell separation chip which comprises the upper plate and the lower plate containing the cell mixture. Further, after the necessary cells have been separated, the homogenization process can be performed to additionally remove unnecessary cells.

1. A cell separation apparatus, comprising:
a lower plate provided with a cell mixture holding portion, in which a cell mixture containing specific cells tagged with magnetic carriers is accommodated in an upwardly convex shape, at a top surface thereof;
an upper plate positioned above the lower plate to face each other and to adsorb the cell mixture accommodated in the cell mixture holding portion of the lower plate into a bottom surface thereof;
a magnetic field applying means positioned on a top surface of the upper plate; and
a gap adjusting means coupled to the upper or lower plate to adjust a gap between the upper and lower plates to be increased or decreased,
wherein the gap between the upper and lower plates is decreased by the gap adjusting means such that the cell mixture accommodated in the cell mixture holding portion is adsorbed in the bottom surface of the upper plate and then formed into a cell mixture layer, and
the gap between the upper and lower plates is increased by the gap adjusting means such that the specific cells moved toward the upper plate by means of a magnetic field applied to the created cell mixture layer through the magnetic field applying means and cells other than the specific cells moved toward the lower plate by means of gravity are divided and then positioned in the bottom surface of the upper plate and the cell mixture holding portion of the lower plate, respectively.

2. The apparatus as claimed in claim 1, wherein a cell mixture adsorbing portion is formed at the bottom surface of the upper plate such that the cell mixture holding portion of the lower plate is positioned to correspond to the cell mixture adsorbing portion of the upper plate.

3. The apparatus as claimed in claim 1, further comprising an upper housing with an open bottom and a lower housing with an open top,
wherein the magnetic field applying means and the upper plate are installed within the upper housing such that the magnetic field applying means is positioned on the top surface of the upper plate,
the lower plate and the gap adjusting means are installed within the lower housing such that the gap adjusting means is coupled to the lower plate, and the lower plate is vertically moved by the gap adjusting means to adjust the gap between the upper and lower plates in a state where the upper housing is coupled with the lower housing such that the bottom surface of the upper plate and the cell mixture holding portion of the lower plate are positioned to face each other.

4. The apparatus as claimed in claim 2, wherein the gap adjusting means comprises a lower plate support formed with a recess for accommodating the lower plate therein at a top side thereof and a bolt-shaped connection at a bottom side thereof, and a lower plate support moving dial having a nut-shaped connection threadedly engaged with the bolt-shaped connection of the lower plate support; and the lower plate support is vertically moved by turning or rotating the lower plate support moving dial.

5. The apparatus as claimed in claim 4, wherein the gap adjusting means further comprises a dial stopper for restricting the rotation of the lower plate support moving means such that the cell mixture layer can be maintained.

6. The apparatus as claimed in claim 5, wherein the dial stoppers are installed on a bottom surface of the lower plate support moving dial and a predetermined portion of the lower housing such that the dial stopper is brought into contact with the bottom surface of the lower plate support moving dial at a position where the rotation of the lower plate support moving dial should be prevented.

7. The apparatus as claimed in claim 3, wherein the gap adjusting means comprises a lower plate support formed with a recess for accommodating the lower plate therein at a top side thereof and a roller at a bottom side thereof, a lower plate support moving bar for vertically moving the lower plate support in such a manner that the roller of the lower plate support is brought into contact with a plurality of steps with different levels decreasing from one side to another side, and bar moving dial having a pinion portion meshed with a rack portion formed on a side surface of the lower plate support moving bar; and the lower plate support is vertically moved as the level of the steps of the lower plate support moving bar brought into contact with the roller of the lower plate support is changed by turning or rotating the bar moving dial.

8. The apparatus as claimed in claim 7, wherein a groove for temporarily restricting a motion of the lower plate support is formed on each of the steps.

9. The apparatus as claimed in claim 8, wherein the lower plate support moving bar includes a lower plate support shaking portion further extending from the step with the highest level and formed with a plurality of grooves.

10. The apparatus as claimed in claim 1, further comprising:
a housing,
wherein the upper and lower plates are installed within the housing such that the bottom surface of the upper plate and the cell mixture holding portion of the lower plate are positioned to face each other; the magnetic field applying means is positioned on the top surface of the upper plate; the gap adjusting means is coupled with the upper plate; and the upper plate is vertically moved by the gap adjusting means to adjust the gap between the upper and lower plates.

11. The apparatus as claimed in claim 10, wherein the gap adjusting means further comprises a stopper for restricting an upward motion of the upper plate support to allow the cell mixture layer to be maintained.

12. A cell separation method, comprising the steps of:
(a) creating a cell mixture containing specific cells tagged with magnetic beads into a cell mixture layer by adjusting a gap between upper and lower plates to be decreased such that the cell mixture which is accommodated in a cell mixture holding portion of the lower plate in an upwardly convex shape can be adsorbed in a bottom surface of the upper plate positioned opposite to the cell mixture holding portion of the lower plate;
(b) moving the specific cells toward the upper plate by applying a magnetic field to the cell mixture layer created in step (a) from the upper plate and simultaneously moving cells other than the specific cells toward the lower plate by means of gravity; and
(c) allowing the specific cells moved toward the upper plate and the other cells moved toward the lower plate in step (b) to be divided and then positioned in the bottom surface of the upper plate and the cell mixture holding portion of the lower plate, respectively, when the cell mixture layer is separated by increasing the gap between the upper and lower plates.

13. The method as claimed in claim 12, further comprising the step of, after step (a), adjusting the gap between the upper and lower plates to maintain a thickness of the cell mixture layer at an optimal cell separation state.

14. The method as claimed in claim 12, further comprising the steps of:
(d1) creating a specific cell mixture layer by decreasing the gap between the upper and lower plates after removing the other cells divided and positioned in the lower plate in step (c) or replacing the lower plate with a new one and then injecting a buffer solution containing no cells in the lower plate;
(e1) homogenizing the specific cell mixture layer by changing the gap between the upper and lower plates repeatedly several time while maintaining the specific cell mixture layer created in step (d1)
(f1) moving the specific cells toward the upper plate by the magnetic field applied to the specific cell mixture layer homogenized in step (e1) from the upper plate and simultaneously moving the other cells in the specific cell mixture layer toward the lower plate by means of gravity; and
(g1) allowing the specific cells moved toward the upper plate and the other cells moved toward the lower plate in step (f1) to be divided and then positioned in the bottom surface of the upper plate and the cell mixture holding portion of the lower plate, respectively, when the specific cell mixture layer is separated by increasing the gap between the upper and lower plates.

15. The method as claimed in claim 12, further comprising the steps of:
(d2) creating an other cell mixture layer by decreasing the gap between the upper and lower plates after removing the specific cells divided and positioned in the upper plate in step (c) or replacing the upper plate with a new one and then additionally injecting a buffer solution containing no cells in the lower plate;
(e2) homogenizing the other cell mixture layer by changing the gap between the upper and lower plates repeatedly several times while maintaining the other cell mixture layer created in step (d2)
(f2) moving the specific cells toward the upper plate by the magnetic field applied to the other cell mixture layer homogenized in step (e2) from the upper plate and simultaneously moving the other cells in the other cell mixture layer toward the lower plate by means of gravity; and
(g2) allowing the specific cells moved toward the upper plate and the other cells moved toward the lower plate in step (f2) to be divided and then positioned in the bottom surface of the upper plate and the cell mixture holding portion of the lower plate, respectively, when the other cell mixture layer is separated by increasing the gap between the upper and lower plates.
